(19) United States
(12) Patent Application Publication
(10) Pub. No.: US 2016/0128602 A1
(43) Pub. Date: May 12, 2016

(54) TISSUE IDENTIFICATION DEVICE AND METHOD

(71) Applicant: MITOS MEDIKAL TEKNOLOJILER SANAYI VE TICARET ANONIM SIRKETI, Maslak Sisli, Istanbul (TR)

(72) Inventors: Mehmet CAYOREN, Istanbul (TR); Mahmut Alp KILIC, Istanbul (TR); Guray Ali CANLI, Istanbul (TR); Murat OZCAN, Istanbul (TR); Ismail KURTOGLU, Istanbul (TR); Resit Ayhan TIGLI, Istanbul (TR); Serkan TEKSOZ, Istanbul (TR); Ibrahim AKDUMAN, Istanbul (TR); Mustafa Tunaya KALKAN, Istanbul (TR)

(21) Appl. No.: 14/899,327

(22) PCT Filed: Jun. 11, 2014
(86) PCT No.: PCT/TR2014/000206

§ 371 Date: Dec. 17, 2015

(30) Foreign Application Priority Data
Jun. 17, 2013 (TR) 2013/07254

(51) Int. Cl. A61B 5/05; A61B 90/70; A61B 5/00

(52) U.S. Cl. CPC A61B 5/0507; A61B 5/05; A61B 5/0077; A61B 90/70; A61B 2090/701; A61B 2560/0223; A61B 2562/0228; A61B 2562/222

(57) ABSTRACT

A tissue identification device for determining the presence and borders of the cancerous tissues includes a measurement probe having a concentric, 2 port coaxial structure and enabling the 1 Port and 2 Port scattering parameters of the target tissue to be measured. An S parameter measurement unit is associated with the measurement probe via an RF/Microwave cable and allows the S parameters of the tissue to be measured. A computer identifies the tissue by means of an application software block and a tissue identification software block therein in line with the measurement results from the S parameter measurement unit. A hand tool carrying the measurement probe thereon during the measurement process operates simultaneously with the computer by being in communication therewith.

### TECHNICAL FIELD

The invention relates to the detection (i.e. identification) of the presence of, and border determination of cancerous tissues on the tissue surface and/or inside the tissue in open or non-open surgeries.

### PRIOR ART

Breast cancer is one of the common diseases of today. One of the basic treatment modalities in breast cancer disease is to remove cancerous tissue by surgery. During surgery, it is the surgeon who decides whether the suspected tissue is cancerous or not, as well as making the decision on the borders of the cancerous tissue. While making this decision, the surgeon uses a pathological method, which is known as “Frozen” in literature, besides his/her own knowledge and experience. This method yields 60-70% accurate results regarding the tissue.

Success criteria of a surgery are to detect cancerous tissue and boerders thereof accurately, and to remove the cancerous tissue completely. In order to be successful in the operation, i.e. to guarantee that the cancerous tissue is fully removed, removing the whole breast, unless really required, is an undesired situation. Therefore, detecting the cancerous tissue and borders thereof as accurately as possible is important in order for the surgery to be successful. In case the surgery fails, the disease cannot be treated and it recurs.

As known in the literature, dielectric permittivity of the cancerous tissue and healthy tissue is different from one another. In this respect, it is a common procedure to detect the presence of cancerous cells by measuring dielectric parameters of the tissues in order to detect the presence of cancerous tissue. However, it is a difficult process to measure dielectric permittivity of the tissue during surgery when it comes to practice. The main reason for that is the environment and bleeding during surgery. Moreover, the tissue is not always exposed during the surgery, and thus it is not possible understand how deep the borders of the cancerous tissues get. Slicing a tissue with a clean surface, or a tissue with tumor, to see what is present therein and controlling over the sliced surfaces, or sectioning for Frozen procedure, are neither practical, nor possible for the surgeon.

In the state of the art, dielectric constant can be measured using open-ended coaxial probe. Said probe measures 1 port scattering parameters (S11). The data obtained from the measurements are analyzed using the known technical methods and the complex dielectric constant of the tissue is determined. However, the use only of 1 port S parameters during dielectric constant determination reduces the accuracy of the determination.

As a result, the aforementioned problems have made it necessary to make a novelty for identifying the presence of cancerous tissue on the tissue surface and/or in the tissue and for determining the borders thereof in open or non-open surgeries in the related technical field.

### BRIEF DESCRIPTION OF THE INVENTION

The present invention relates to a microwave tissue identification device and method developed for eliminating the above disadvantages and providing new advantages in the related technical field.

An object of the invention is to introduce a microwave tissue identification device in order to detect the presence and borders of the cancerous tissues in the deep, in addition to the cancerous tissues on the surface, in open and/or non-open surgeries.

Another object of the invention is to introduce a tissue identification device and method in order to increase the accuracy of the identification regarding whether a tissue is cancerous or not when compared to the known methods.

And another object of the invention is to present a tissue identification device, which is practical and ergonomic while being used in open and/or non-open surgeries and, which is directive (i.e. guiding) and informative for the surgeon who will perform the surgery. In order to achieve all the objectives that have been mentioned above and will be further understood from the following description, the present invention relates to a tissue identification device for determination (i.e. identification) of the presence/absence and borders of the cancerous tissues. Said tissue identification device is characterized in comprising;
a measurement probe having a concentric, 2 Port coaxial structure, which allows measuring 1 Port and 2 Port scattering parameters of the target tissue,
an S parameter measurement unit, which is associated with said measurement probe by means of RF/Microwave cable and allows measuring S parameters of the tissues, and
a computer, which identifies the tissue by way of the application software block and tissue identification software block that it comprises, in line with the measurement results from S parameter measurement unit.

Tissue identification method performed using said tissue identification device and aiming to determine the presence and borders of the cancerous tissues basically comprises the process steps of;
a) contacting the measurement probe with the target tissue,
b) measuring 1 port and 2 port scattering parameters of the tissue in a frequency band thanks to the concentric, 2 port coaxial structure of the measurement probe by means of S parameter measurement unit,
c) transferring the measurement results to the application software block of the computer, and to the tissue identification software block by the application software block,
d) evaluating the measurement results by means of the tissue identification software block and performing tissue identification.

In order for the embodiment of the invention and the advantages thereof, together with additional components, to be better understood, it should be evaluated together with the figures, the descriptions of which are given below.

### BRIEF DESCRIPTION OF THE FIGURES

FIG. 1 shows the block diagram of the tissue identification device according to the invention.

FIGS. 2 and 3 show the probe structure of the tissue identification device according to the invention.

FIG. 4 shows the measurement probe, together with the equipment with which it is used in the measurement of deep tissues.

FIG. 5 shows the view of the measurement probe while measuring deep tissues.

FIG. 6 shows the perspective view of the hand tool together with which the measurement probe is used.

### REFERENCE NUMERALS

10 Tissue Identification Device

11 Computer
111 Screen
112 Application Software Block
113 Tissue Identification Software Block

12 Hand Tool
121 Holder
122 Measurement Button
123 Micro-control Card
124 Hand Tool Embedded Software Block
125 Screen
126 Camera

13 Hand Tool Computer Connection

14 Camera Computer Connection

15 RF-Microwave cable

16 Measurement Probe
161 Guide
162 Elastic Stopper
163 Port 1 Conductive Live End
164 Dielectric Material Type 1
165 Port 1 and Port 2 Conductive Ground Connection
166 Dielectric Material Type 2
167 Port 2 Conductive Live End
168 Non-conducting Cover

17 S Parameter Measurement Unit

18 S Parameter Measurement Unit-Computer Connection

19 Calibration and Sterilization Unit
191 Pure Water Tank
192 Temperature Control Circuit
193 Thermoelectric Temperature Control Unit
194 Temperature Indicator
195 Liquid Sterilization Tank
196 Short-Circuit Calibration Tank
197 Cleaning and Drying Tank with Air
198 Open-Circuit Calibration Housing
20 Energy Module

21 Energy Supply

22 Power Distribution Unit

23 On/Off Button

### DETAILED DESCRIPTION OF THE INVENTION

In this detailed description, the tissue identification device (10) according to the invention will only be described in order for the subject matter to be better understood by way of illustrations, without any limitations.

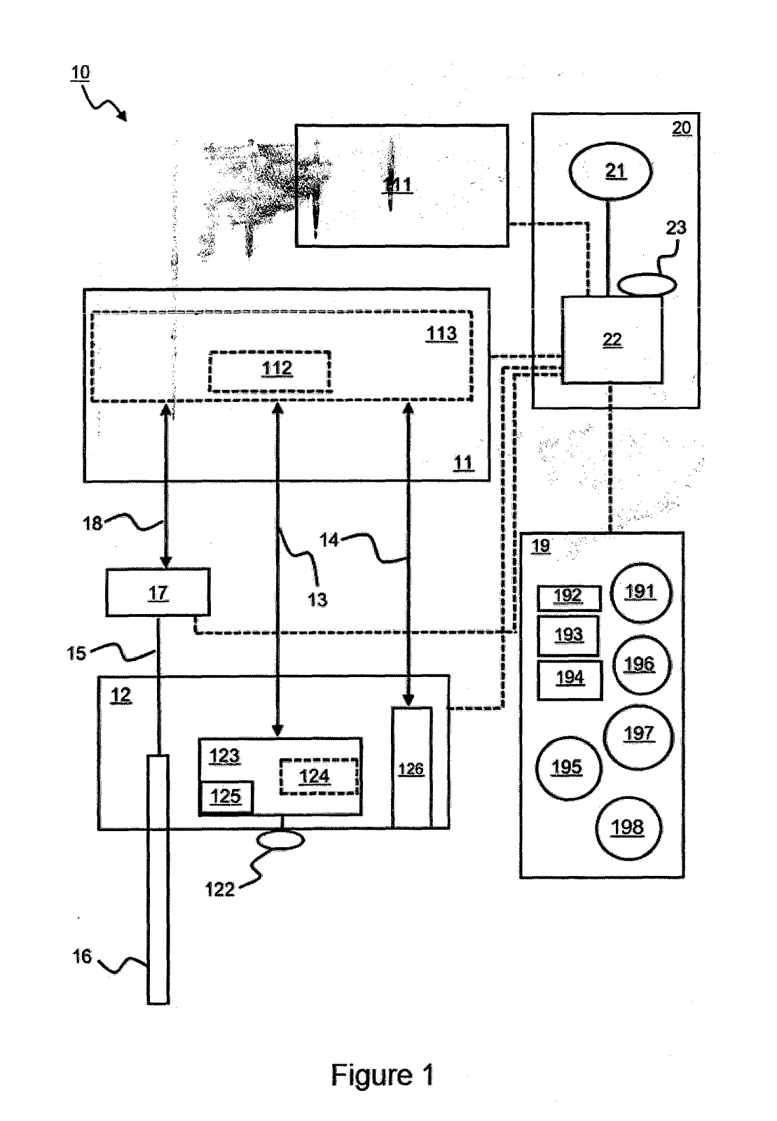

FIG. 1 is the block diagram of the tissue identification device (10) according to the invention. The tissue identification device (10) basically comprises;
a computer (11) comprising the required software blocks for performing the measurement process,
a measurement probe (16), which operates in association with said computer (11) and is configured for determining the dielectric constant of the tissue in open and/or non-open surgeries, and
an S parameter measurement unit (17), which is connected with said measurement probe (16) and computer (11) and which measures scattering parameters (S parameters) of the tissue by means of the measurement probe (16) and transfers the measurement results to the computer (11).

In addition to the above components, the tissue identification device (10) according to the invention comprises a hand tool (12), which carries the measurement probe (16) thereon and operates in association with the computer (11). Moreover, a calibration and sterilization unit (19), which comprises the required components for providing calibration and sterilization of the measurement probe (16) during surgery is also provided. Also provided herein is an energy module (20) for providing the required power for the operation of the computer (11), hand tool (12), calibration and sterilization unit (19), which are disposed in the tissue identification device (10). Said energy module (20) is activated by on/off button of the device (10). The energy module (20) comprises an energy supply (21), which enables the device (10) to be connected to a power source, e.g. electric supply system, battery, as well as comprising a power distribution unit (22) for distributing the energy to the components comprised by the device (10).

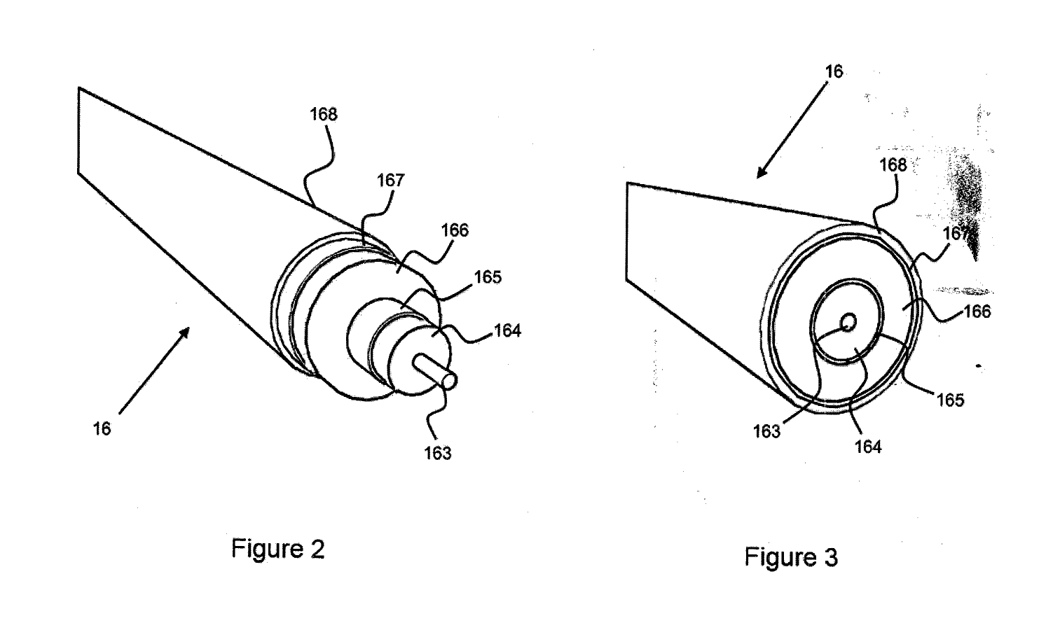

The measurement probe (16) is the concentric, 2 port coaxial measurement probe (16); and in FIG. 2, the layer structure of the probe (16) is shown. The coaxial structure forming the 1st port of the measurement probe (16) includes the port 1 conductive live end (163); the dielectric material Type 1 (164) enclosing said port 1 conductive live end (163); and Port 1 and Port 2 conductive ground connection (165) enclosing said dielectric material Type 1 (164). The port 1 conductive live end (163) is a metal rod, which is provided in the center of the whole configuration. The coaxial structure forming the 2nd port thereof, on the other hand, includes said Port 1 and Port 2 conductive ground connection (165); the dielectric material Type 2 (166) enclosing the Port 1 and Port 2 conductive ground connection (165); and the port 2 conductive live end (167) enclosing said dielectric material Type 2 (166). Said port 2 conductive live end (167) is a metal cover. The whole configuration described above is provided inside a non-conducting cover (168). This layer structure of the measurement probe (16) allows the measurement of 2 port S parameters (S11, S12, S21, S22), besides 1 port S parameters of the tissue, the complex dielectric constant of which is desired to be determined.

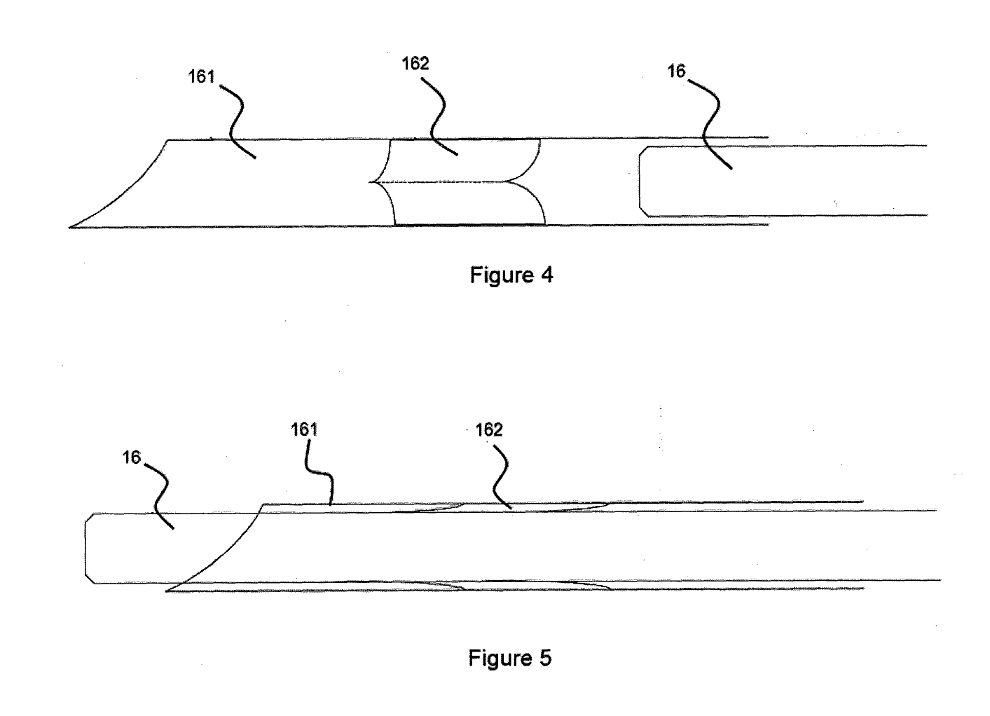

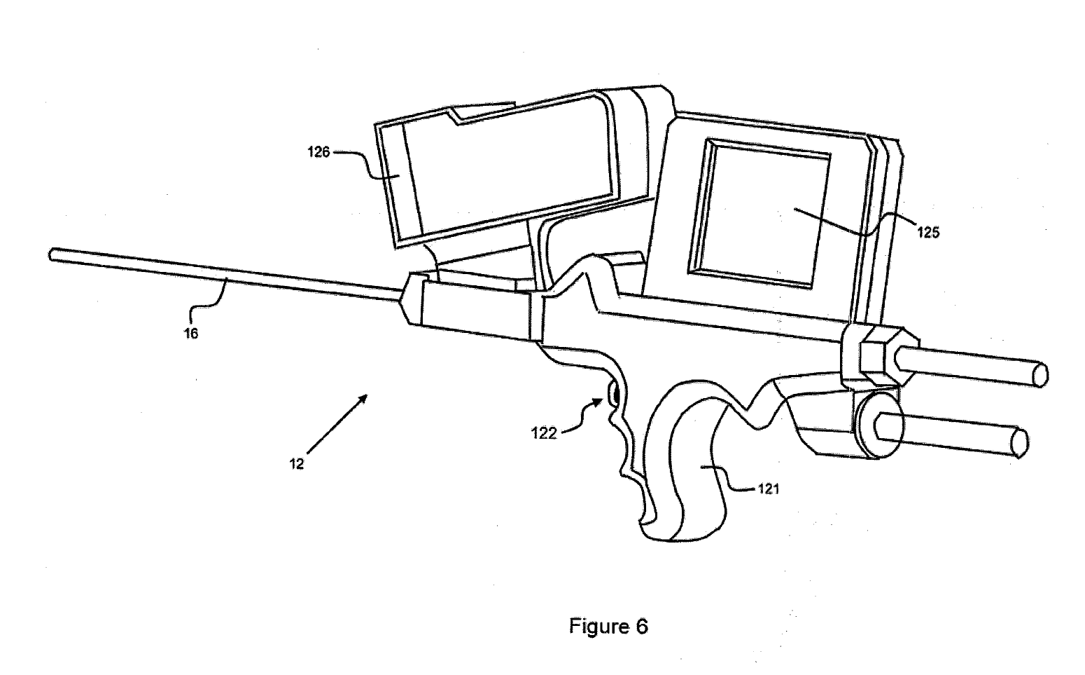

FIG. 4 shows the use of measurement probe (16), together with the equipment with which it is used for measuring the deep tissue, in open and/or non-open surgeries. Accordingly, the measurement probe (16) is disposed in a guide (161) and it is immersed in the tissue through said guide (161). The guide (161) enables the measurement probe (16) to be directed without contacting any other point than/until the target tissue. There is an elastic stopper (162) inside the guide (161). Said elastic stopper (162) is opened upon being pushed for contacting the measurement probe (16) with the tissue and allows the measurement probe (16) to contact/touch the tissue. Hence, the elastic stopper (162) allows the measurement probe (16) to remain clean during its movement inside the guide (161).

The measurement probe (16) is preferably thinner than 1.5 mm diameter. The guide (161), on the other hand, has a width such that the measurement probe (16) will pass therethrough. Thus, the measurement probe (16) can easily operate inside the guide (161); and measurement beneath the tissue, besides over tissue measurements, can be performed in open or non-open surgeries, e.g. biopsy.

The S parameter measurement unit (17) measures 1 port or 2 port S parameters of the tissue by means of the measurement probe (16) connected thereto via RF/Microwave cable (15). The measurement of S parameters is performed in a frequency band range. This frequency band preferably ranges from 100 MHz to 6 GHz. In this way, dielectric permittivity of the tissue are expressed as frequency function and frequency-related changes in the tissue are assessed for identifying the tissue. Hence, a more accurate result will be achieved compared to the result obtained from a single frequency. S parameter measurement unit (17) transfers the measurement results to the computer (11) through the S parameter measurement unit-computer connection (18), said connection being provided between the computer (11) and itself.

The measurement probe (16) is used by being attached to the hand tool (12) during surgeries. The overall view of the hand tool (12) is given in FIG. 6. The hand tool (12) is preferably configured in the form of a gun and comprises a holder (121) having a measurement button thereon (122). Also provided in the hand tool (12) are; a camera (126) taking the image of the tissue to be measured and a screen (125) where the required information for directing and informing the user during measurement process is indicated.

The hand tool (12) is associated with the computer (11) by way of hand tool-computer connection (13). The hand tool (12) comprises a micro-control card (123) and a hand tool embedded software block (124) running over said micro-control card (123). The management of the hand tool (12), as well as its communication with the computer (11), is provided by the micro-control card (123) and the hand tool embedded software block (124). When the user pushes the measurement button (122) on the holder (121) during the measurement process, the micro-control card (123) directs the information determined by the hand tool embedded software block (124) to the computer (11). The data from the computer (11) are sent to the micro-control card (123) and indicated on the hand tool (12) screen (125) by the hand tool embedded software block (124).

The computer (11) comprises an application software block (112), which processes the data transferred by the S parameter measurement unit (17), hand tool (12), and hand tool (12) camera (126) and manages the measurement process in line with this data. Prior to the surgery, the information on patient, surgery, and potential diagnosis are saved in a database by means of the application software block (112). The measurement results from the S parameter measurement unit (17) and the images taken by the camera (126) are transferred to a tissue identification software block (113) disposed in the computer (11) via the application software block (112). Said tissue identification software block (113) processes S parameters by using the predetermined algorithm thereof and allows the determination of the dielectric constant of the tissue. The determined result and camera (126) images are shown on the computer (11) screen (111). The required information for directing and informing the surgeon who will perform the surgery during the measurement process are shown on the computer (11) screen (111) and hand tool (12) screen (125) simultaneously. It is the application software block (112), which determines what information will be shown on the screen (111) at what process step.

When supply voltage is given to the tissue identification device (10) and when the surgeon pushes the measurement button of the hand tool (12), the application software block (112) automatically starts the calibration and sterilization process of the measurement probe (16). This process is performed in the calibration and sterilization unit (19). This unit comprises;
a pure water tank (191),
a thermoelectric temperature control unit (193) and a temperature control circuit (192), which together allow the temperature of the pure water in said pure water tank (191) to remain at a certain range by controlling it,
a temperature indicator (194) showing/indicating water temperature,
a liquid sterilization tank (195) where the measurement probe (16) is sterilized,
a cleaning and drying tank with air (197) where the measurement probe (16) is immersed after the liquid sterilization tank (195), and
a microwave short circuit calibration housing (196).

The surgeon pushes the measurement button (122) while passing/switching from one process step to another during calibration and sterilization process, thereby making the tissue identification device (10) pass to the next step. Thus, surgeon can perform measurement and calibration processes only by pushing the measurement button (122) disposed on the hand tool (12) and by observing the screen (125) provided on the hand tool (12), and hence s/he does not have any difficulty in concentrating during the surgery.

In line with the above explanations, the main operations/processes performed by the tissue identification device (10) according to the invention are as follows; calibration and sterilization of the measurement probe (16); measurement of S parameters of the target tissue; evaluation of measurement results; and identification of the tissue.

Switching on the tissue identification device (10) is performed as follows: First, the device (10) is connected to a power source, e.g. electric supply system, battery, by means of the energy supply (21). The device (10) is activated by means of the on/off button on the device (10). The required energy for the operation of all units of the device (10) is transferred by the power distribution unit (22). Activation duration of the device (10) is shown on the computer screen (111).

The calibration and sterilization unit (19) starts to operate with the energy supply (21). The thermoelectric temperature control unit (193) and the temperature control circuit (192) start to stabilize the pure water in the pure water tank (191) at a temperature of 25° C. +/−0.5° C. The temperature indicator (194) displays the current temperature of the pure water. After the surgeon sees on the computer screen (111) that the switching on process of the device (10) is completed, s/he checks the temperature indicator (194) and controls whether the temperature of the pure water is 25° C. Since the device (10) has just been switched on, the application software block (112) directly starts the calibration and sterilization process. At this stage, the operations to be performed by the surgeon are shown on the computer screen (111) and the hand tool (12) screen (125). The following processes are respectively performed at calibration and sterilization stage:
immersing the measurement probe (16) in the liquid sterilization tank (195),
immersing the measurement probe (16) in the cleaning and drying tank with air (197),
immersing the measurement probe (16) in microwave open-circuit calibration housing (198),
immersing the measurement probe (16) in microwave short-circuit calibration housing (196), and
immersing the measurement probe (16) in the pure water tank (191).

After the surgeon finishes a process step, s/he pushes the hand tool (12) measurement button (122), thereby the information on the next process step being shown on the hand tool (12) screen (125) and computer screen (111).

Upon completion of the calibration and sterilization process, the device (10) gets ready for the measurement process. The measurement method of the tissue identification device (10) according to the invention is based on the principle of measuring the 1 port or 2 port scattering parameters of the tissue in a certain frequency band, and of determining the complex dielectric permittivity of the tissue by the algorithm defined in the tissue identification software block (113) in line with these measurements.

The basic process steps of the measurement process are as below, although there are some different process steps depending on whether the surgery is open or non-open:
the surgeon grabs the hand tool (12) from the holder (121) and makes the measurement probe (16) contact with the tissue, the dielectric constant of which s/he wishes to determine,
s/he pushes the measurement process (122) on the holder (121) and starts the measurement process,
the camera (126) provided on the hand tool (12) takes the image of the tissue being measured and transfers it to the application software block (112) via the camera-computer connection (14),
the S parameter measurement unit (17) measures S parameters of the tissue in a frequency band (preferably 100 MHz-6 GHz) by means of the measurement probe (16) connected thereto via RF/microwave cable (15),
S parameters having been measured are transferred to the computer (11) via S parameter measurement unit-computer connection (18),
S parameters and the tissue images are transferred to the tissue identification software block (113) and tissue identification is made using a predetermined tissue identification algorithm,
the measurement result is transferred to the application software block (112), and the tissue identification result and the image of the measured tissue are shown on the computer (11) screen (111) and on the hand tool (12) screen (125) by the application software block (112),
after this process, the device (10) becomes ready for subsequent measurements, and
the application software block (112) stores the number of the measurements in the memory thereof, and when necessary, warns the surgeon through the hand tool (12) screen (125) and computer (11) screen (111) to start the calibration and sterilization process.

During an open surgery, the surgeon opens the breast first. In case that the tissue to be measured is on the surface, the surgeon performs measurement process by repeating the above process steps.

If the tissue identification device (10) is to be used in an open surgery where the tissue to be measured is in the deep, or in a non-open surgery such as biopsy, the measurement probe (16) is immersed in the tissue through said guide (161). When the tip of the guide (161) contacts with the tissue desired to be measured, the surgeon pushes the measurement probe (16) and opens the elastic stopper (162) inside the guide (161), and then makes the measurement probe (16) contact with the tissue. Afterwards, the above measurement steps are performed and the dielectric constant of the tissue is identified.

1. A tissue identification device for identification of the presence/absence and borders of the cancerous tissues, characterized in comprising:
a measurement probe having a concentric, 2 Port coaxial structure, which allows measuring 1 Port and 2 Port scattering parameters of the target tissue,
an S parameter measurement unit, which is associated with said measurement probe by means of an RF/Microwave cable and allows measuring S parameters of the tissues, and
a computer which identifies the tissue by way of an application software block and tissue identification software block that it comprises, in line with the measurement results from S parameter measurement unit.

2. The tissue identification device (10) according to Clam 1, characterized in comprising a hand tool, which carries said measurement probe thereon during the measurement process, operates simultaneously with the computer by being in communication therewith, and is suitable for being used with a single hand, either by right or left hand.

3. The tissue identification device according to claim 2, characterized in comprising a calibration and sterilization unit, which comprises the required components for providing calibration and sterilization of the measurement probe.

4. The tissue identification device according to claim 1, characterized in that the measurement probe comprises;
a port 1 conductive live end provided in the center; a dielectric material Type 1 enclosing said port 1 conductive live end; and a Port 1 and Port 2 conductive ground connection enclosing said dielectric material Type 1, which form the 1st port thereof; and
said Port 1 and Port 2 conductive ground connection; dielectric material Type 2 enclosing the ground connection; and port 2 conductive live end enclosing the dielectric material Type 2, which form the 2nd port thereof.

5. The tissue identification device according to claim 4, characterized in comprising a non-conducting cover enclosing said 1st and 2nd ports of the measurement probe.

6. The tissue identification device according to claim 1, characterized in comprising a guide which enables the probe to be directly directed to the target tissue by guiding it inside the tissue during the measurement of dielectric permittivity of deep tissues.

7. The tissue identification device according to claim 1, characterized in comprising a measurement button which, when pushed thereon, transfers the information determined by the hand tool embedded software block of the micro-control card to the computer.

8. The tissue identification device according to claim 2, characterized in comprising a screen on which measurement results are shown, which enables the hand tool to be directed and informed about the measurement process during the surgery, and which is located on said hand tool.

9. The tissue identification device according to claim 6, characterized in comprising an elastic stopper, which covers the end of the measurement probe by being located inside the guide and is opened upon the contact of the measurement probe with the target tissue.

10. The tissue identification device according to claim 2, characterized in comprising a hand tool embedded software block and a micro-control card, which together provide the management of the hand tool and the coordination thereof with the computer.

11. The tissue identification device according to claim 2, characterized in that the hand tool comprises a camera, which takes the images of the measured tissue and transfers these images to the computer.

12. The tissue identification device according to claim 2, characterized in that said hand tool comprises a holder.

13. The tissue identification device according to claim 3, characterized in comprising a pure water tank, which is provided in said calibration and sterilization unit and comprises pure water therein.

14. The tissue identification device according to claim 13, characterized in that said calibration and sterilization unit comprises a thermoelectric temperature control unit and a temperature control circuit, which together allow the temperature of the pure water in said pure water tank to remain at a certain range by controlling it.

15. The tissue identification device according to claim 3, characterized in that said calibration and sterilization unit comprises a temperature indicator showing the temperature of pure water.

16. The tissue identification device according to claim 3, characterized in that said calibration and sterilization unit comprises a liquid sterilization tank where the measurement probe is sterilized and a cleaning and drying tank with air where the measurement probe is immersed after the liquid sterilization tank.

17. The tissue identification device according to claim 3, characterized in that said calibration and sterilization unit comprises a microwave short-circuit calibration housing.

18. The tissue identification device according to claim 3, characterized in that said calibration and sterilization unit comprises a microwave open-circuit calibration housing.

19. A tissue identification method performed using said tissue identification device according to claim 1 and aiming to determine the presence and borders of the cancerous tissues, characterized in comprising the process steps of;
a) contacting the measurement probe with the target tissue,
b) measuring 1 port and 2 port scattering parameters of the tissue in a frequency band thanks to the concentric, 2 port coaxial structure of the measurement probe by means of S parameter measurement unit,
c) transferring the measurement results to the application software block of the computer, and to the tissue identification software block by the application software block,
d) evaluating the measurement results by means of the tissue identification software block and performing tissue identification.

20. The tissue identification method according to claim 19, characterized in that said frequency band ranges from 100 MHz to 6 GHz in step b.

21. The tissue identification method according to claim 19, characterized in that the image of the tissue is taken by the camera of the hand tool and is transferred to the application software block together with S parameters in step b.

22. The tissue identification method according to claim 21, characterized in that the measurement results and tissue images are shown on the screen of the computer by the application software block.

23. The tissue identification method according to claim 19, characterized in that the application software block activates the calibration and sterilization unit for calibration and sterilization while switching on the tissue identification device.

24. The tissue identification method according to claim 19, characterized in that the application software block stores the number of measurements in the memory thereof so that calibration and sterilization process will be started when necessary during the process period of the measurement probe.

25. The tissue identification method according to claim 23, characterized in comprising, during calibration and sterilization, the process steps of
immersing the measurement probe in a liquid sterilization tank, immersing
the measurement probe in a cleaning and drying tank with air,
immersing the measurement probe in a microwave open-circuit calibration housing,
immersing the measurement probe in a microwave short-circuit calibration housing, and
immersing the measurement probe in a pure water tank.

26. The tissue identification method according to claim 25, characterized in that, for passing from one process step to another, the measurement button of the hand tool is pushed and the application software block and hand tool embedded software are activated accordingly during the calibration and sterilization process.

27. The tissue identification method according to claim 19, characterized in comprising the following process steps for making measurement through deep tissues in open and/or non-open surgeries;
immersing a guide including the measurement probe in the tissue,
pushing the measurement probe when the end of the guide contacts with the target tissue,
opening of the elastic stopper provided in front of the measurement probe upon pushing of the latter,
performing the measurement process upon contact of the measurement probe with the tissue.
